(19) United States
(12) Patent Application Publication
(10) Pub. No.: US 2026/0325058 A1
(43) Pub. Date: Oct. 22, 2026

(54) METHOD FOR DETERMINING A PROTECTION FACTOR OF A SKIN PROTECTANT

(71) Applicant: COURAGE + KHAZAKA ELECTRONIC GMBH, Koln (DE)

(72) Inventors: Carina REBLE, Koln (DE); Georg WIORA, Koln (DE)

(21) Appl. No.: 19/115,646

(22) PCT Filed: Sep. 27, 2023
(86) PCT No.: PCT/EP2023/076801

§ 371 Date: Mar. 26, 2025

(30) Foreign Application Priority Data
Sep. 27, 2022 (DE) 10 2022 124 831.3
Nov. 21, 2022 (DE) 10 2022 130 758.1
Mar. 27, 2023 (DE) 10 2023 107 705.8
Sep. 19, 2023 (DE) 10 2023 125 291.7

(51) Int. Cl. A61B 5/00; G01N 21/31; G01N 21/33

(52) U.S. Cl. CPC A61B 5/441; G01N 21/33; G01N 2021/3155; G01N 2201/121

(57) ABSTRACT

The invention relates to a method for determining a protection factor of a skin protectant, said method comprising the steps of emitting radiation from a radiation source of a protection factor evaluation system, detecting remitted radiation by a detector unit of a protection factor evaluation system, transmitting data on the remitted radiation to an analysis unit, and evaluating the protection factor in an evaluation wavelength range, wherein, during the evaluation of the protection factor, a correction function is taken into consideration, the correction function describing skin type-dependent differences.

The invention relates to a method for determining a protection factor of a skin protectant, said method comprising the steps of emitting radiation from a radiation source of a protection factor evaluation system, detecting remitted radiation by a detector unit of a protection factor evaluation system, transmitting data on the remitted radiation to an analysis unit, and evaluating the protection factor in an evaluation wavelength range, wherein, during the evaluation of the protection factor, a correction function is taken into consideration, the correction function describing skin type-dependent differences.

### PRIOR ART

The methods approved to date by the authorities of the European Union (EU) and the U.S. Food and Drug Administration (FDA) for determining the SPF (Sun Protection Factor) are all harmful to the test subjects involved by causing erythema, that is, an inflammatory reaction of the skin caused by light (COLI PA-15 European Cosmetic, Toiletry and Perfumery Association: Colipa SPF Test Method 94/289, 1994; ISO standards 24442, 24443, 24444). Therefore, both the FDA and the EU have repeatedly pointed out that future research activities must be directed towards new methods for characterizing the protective efficacy of sunscreen products in order to avoid long-term effects on the test subjects (European Commission, 20 Standardisation Mandate Assigned To CEN Concerning Methods For Testing Efficacy Of Sunscreen Products, M/389 EN, Brussels, 12 Jul. 2006).

This invention is intended to accomplish this task. The existing methods are defined in various sources:

Methods defined in standards and regulations:

a. ISO 24444 defines a method for in vivo determination of the SPF. The method is based on generating erythemas on the skin of test subjects by radiation in the UVB range. Therefore, the method is harmful to the test subject.

b. ISO 24443 defines an in vitro method for the determination of the UVA protection factor (UVAPF). The sunscreen is applied to a plastic plate so that a transmission spectrum of the sunscreen can be measured. Due to uncontrollable fluctuations in the procedure, the transmission spectrum is adapted to the result of the erythema test according to ISO 24444 by scaling and is therefore dependent on its implementation. The plastic plate used has a roughened surface and is an unrealistic skin model.

c. ISO 24442 defines an in vivo method in which the UVA protection factor is determined using the minimum UVA dose required to produce irreversible pigmentation (suntan) of the skin. This method also causes a change in the test subject's skin.

### Patented Methods

DE 198 28 497 A1 describes a method in which, as in ISO 24444, erythemas are induced in test subjects by UV irradiation of the skin. In contrast to ISO 24444, erythemas are detected by reflection spectroscopy. The method is therefore also damaging. The optical effect (protection) of the sunscreen is not determined by direct optical measurements, but by a biological reaction of the body.

DE 10 2004 020 644 A1 describes a method in which the generation of radicals by UV exposure in vivo is quantitatively measured by means of electron spin resonance (ESR). Here, too, the optical effect of the sunscreen is only captured indirectly. In addition, the measurement by ESR is technically complex and requires relatively large, stationary devices (tabletop devices). These are also sensitive to interference from high-frequency radiation or rapid temporary changes in the magnetic field, such as those caused by electrical switching processes.

To determine the label SPF of topically applied sunscreens in vivo, test methods such as ISO 24444, the FDA guideline, or the Australian standard are used worldwide. The basis of all these methods is the induction of an erythemal skin reaction by irradiating the skin with UV light. This is necessary to determine the minimum erythemal dose of untreated (MEDu) and product-treated skin (MEDp). Reliable in vitro methods in which human skin is replaced by synthetic substrate carriers are not available for SPF determination.

Monochromatic devices are known that use conventional xenon lamps and are therefore expensive to purchase and operate. Built-in monochromators measure different wavelengths one after the other, which is disadvantageous when the test subjects are moving. Polychromatic devices likewise use xenon lamps. The in vivo measured value is weighted using a filter so that it matches the in vivo UVA-PF. Multi-LED devices for testing institutes are another option, but they are significantly larger and more expensive due to the spectroscopic detection.

It is therefore an object of the invention to provide a method for determining a protection factor of a skin protectant, which method provides high-quality analysis results and at the same time can be carried out quickly and easily.

The object is also achieved by means of the method according to the invention for determining a protection factor of a skin protectant. Advantageous embodiments of the invention are provided in the dependent claims.

The method according to the invention for determining a protection factor of a skin protectant has four method steps: In the first method step, radiation is emitted from a beam source of a protection factor evaluation system. The beam source generates electromagnetic radiation with an irradiation wavelength between the blue spectral range (wavelength from about 400 nm to 500 nm) and the UV range (280 nm to 400 nm).

The wavelength ranges are defined as follows:
<320 nm (UVB) with <0.1% of the total UV intensity,
320 nm to 340 nm (UVA II), 8% to 20% of the total UVA intensity
340 nm to 400 nm (UVA I), 80% to 92% of the total UVA intensity
400 nm to 500 nm, blue light

In the context of this document, the beam source is a technical device for generating electromagnetic radiation. A beam source is therefore not an optical element for guiding, deflecting or changing the intensity and/or wavelength of electromagnetic radiation. A beam source is therefore not a light guide, grating, prism, or filter, for example.

In the second method step, remitted radiation is detected by a detector unit of a protection factor evaluation system. The ratio of the intensities of the remitted radiation to the radiation coupled into the measuring body is a measure of the protective ability of the protectant. The detection wavelength, like the irradiation wavelength, preferably comprises a wavelength range, wherein the wavelength range of the detection wavelength preferably lies within the range of the irradiation wavelength or comprises the entire range of the irradiation wavelength.

In the third method step, data of the remitted radiation is transferred to an evaluation unit. The evaluation unit is a computer, for example, and has a suitable computer program.

In the fourth method step, the protection factor is evaluated in an evaluation wavelength range. The evaluation wavelength is the wavelength for which the protection factor is determined. The evaluation wavelength is different from the wavelength range of the irradiation wavelength and/or the detection wavelength. The evaluation wavelength, like the irradiation wavelength and the detection wavelength, is preferably a wavelength range, wherein the wavelength range of the evaluation wavelength comprises at least the wavelength range of one of the irradiation wavelength and the detection wavelength.

The protection factor (SF) is a scientific measure and indicates how much lower the risk of skin damage is through the use of a protectant. This factor focuses on the time it takes for UVB rays to penetrate a protectant and cause the skin to turn red (minimal erythema, MED) compared to the time it takes when no protectant is present. The dose of solar radiation required to cause reddening of the skin is divided by the dose required to cause reddening without any protectant. This calculation is based on the application of 2 milligrams of protectant per square centimeter of skin surface. Currently, the SF of protectants is determined by in vivo irradiation using a sun simulator (ISO 24444:2010 ‘Cosmetics—Sun protection test methods—In vivo determination of the sun protection factor (SPF),’ which represents the current state of the art. The basis for current testing of protectants is that test subjects are irradiated before and after the application of protectants.

According to the invention, a correction function is taken into consideration when evaluating the protection factor, wherein the correction function describes differences depending on the skin type. This allows a more precise determination of the protective ability of a protectant, and the determined protective ability is comparable for different skin types.

In a further development of the invention, the correction function outputs a corrected value SF_{corr} or UVA-SF_{corr}. In a further embodiment of the invention, the correction function is F(SF)=SF__{corr} or F(UVA-SF)=UVA-SF__{corr}. In a further design of the invention, the correction function makes the measured values of different skin types comparable through the corrected values. The protective ability of the protectant can therefore be determined independently of the skin type of a test subject. In a further embodiment of the invention, the correction function F is a linear factor or an exponential function.

In a further embodiment of the invention, the correction function has a constant C, wherein C depends on the skin type or the ITA° value. Calculation of the individual typology angle (ITA) based on spectrophotometric measurements is used to classify skin types into 6 physiologically relevant groups: very fair, light, medium, brown, brown, and dark.

The determined ITA° values reflect a person's sensitivity to UV radiation and consequently the sensitivity of the skin to solar radiation and skin damage such as pigment changes, cancer, and aging.

The CIELAB color space (also known as L*a*b*) is a color space defined by the International Commission on Illumination (CIE). It expresses color on three axes. In addition, the individual typology angle ITA° is automatically calculated from L* (brightness) and b* (yellow color spectrum). ITA° is a recognized parameter for objectifying skin color to predict the biological consequences of UV irradiation on the skin. The difference along the yellow-blue axis (parameter b*) and along the light-dark axis (parameter L*) determines the intensity of skin pigmentation. In a person with light skin color, ITA° is expected to have a higher positive value than in a person with a darker skin type.

The ITA° value is calculated using the following equation:

ITA°=(arctan(L*−50)/b*))*180/p
where L* stands for the luminance in the range from black (0) to white (100) and b* in the range from yellow to blue (12). The higher the ITA° value, the lighter the skin. The resulting ITA° value classifies skin types into 6 physiologically different categories: ITA°>55° “very light”, 55°>ITA°>41° “light”, 41°>ITA°>28° “intermediate”, 28°>ITA°>10° “tanned”, 10°>ITA°>−30° “brown”, −30°>ITA° “dark.”

In a further embodiment of the invention, the skin type-dependent differences are determined by a measurement. In a further aspect of the invention, the skin type-dependent differences are determined by a reflection measurement. For this purpose, a probe head is applied to the skin of a test subject. The probe head emits white light. The white light is scattered in all directions by the skin surface and partially penetrates the skin surface and is reflected back. This reflected light is measured by the probe head. The remitted spectrum is adjusted to the DIN standard values using a special color matrix and expressed as XYZ (tristimulus). The measured color is output as an ITA° value in the CIELAB color space.

In a further embodiment of the invention, the emitted radiation comprises an irradiation wavelength between 280 nm and 2000 nm, preferably 280 to 800 nm, particularly preferably the range from 280 to 500 nm. The beam source generates electromagnetic radiation with an irradiation wavelength between the blue spectral range (wavelength from about 400 nm to 500 nm) and the UV range (280 nm to 400 nm).

In a further design of the invention, the evaluation wavelength range is different from the irradiation wavelength range. The evaluation wavelength is the wavelength for which the protection factor is determined. The evaluation wavelength is different from the wavelength range of the irradiation wavelength and/or the detection wavelength. The evaluation wavelength, like the irradiation wavelength and the detection wavelength, is preferably a wavelength range, wherein the wavelength range of the evaluation wavelength comprises at least the wavelength range of one of the irradiation wavelength and the detection wavelength.

In a further advantageous design of the invention, the protection factor of the protectant is evaluated from the remitted radiation and the transmission spectrum. Due to its high absorption properties, human skin does not emit enough UVB radiation to measure the absorption spectrum of the applied product in the UVB range. It is therefore necessary to record the absorption spectrum of the test material in the UVB part of the spectrum (280-320 nm) separately using another technique-a transmission spectrum. A transmission spectrum is determined based on the UV transmittance of protective films in vitro. The substrates to which the protective films are applied only approximately reproduce the inhomogeneous surface structure of human skin, such as polymethyl methacrylate (PMMA) sheets with a rough surface according to ISO 24443. The transmission spectrum data includes intensity versus wavelength in preferably digitized format.

In a further embodiment of the invention, the data of the transmission spectrum for determining the protection factor is read from a database of the evaluation unit. The evaluation unit has a database in which data from different transmission spectra can be stored, or is connected to such a database.

In a further design of the invention, the data of the transmission spectrum is in silico data. The data of the transmission spectrum is therefore neither determined in vivo on a test subject nor in vitro according to ISO 24443, but is estimated or determined mathematically. If the properties of the filter substances of a protectant are known, the transmission can be calculated and simulated. Based on the simulated transmission, the sun protection factor and all quantities that characterize the protection factor can be calculated.

In a further development of the invention, the wavelength range of the transmission spectrum comprises the evaluation wavelength. The evaluation of the protection factor of the protectant is carried out from the in vivo remitted radiation and the in silico transmission spectrum. Due to its high absorption properties, human skin does not emit enough UVB radiation to measure the absorption spectrum of the applied product in the UVB range. It is therefore necessary to record the absorption spectrum of the test material in the UVB part of the spectrum (280-320 nm) separately using another technique. The approach used in this document uses the in vivo evaluation of the absolute UVA absorption spectrum as measured by an in vivo measurement with the use of a calculated in silico transmission spectrum to determine the protection factor of the protectant for a user. The evaluation and hybridization of an in vivo remission spectrum with an in silico transmission spectrum is carried out to obtain a complete UV spectrum so that the protection factors can be calculated according to the formulas of the valid standard (ISO 24443). For this purpose, the wavelength range of the transmission spectrum includes the evaluation wavelength, which preferably comprises the range from 280 nm to 500 nm.

In a further embodiment of the invention, the evaluation wavelength comprises the wavelength range from 280 nm to 2000 nm, preferably the wavelength range from 280 nm to 800 nm or particularly preferably the wavelength range from 280 nm to 500 nm. In a further development, the evaluation wavelength comprises a wavelength range from 400 nm to 500 nm and preferably from 400 nm to 450 nm. Preferably, the wavelength range of blue light, which borders on the UVA range (up to 400 nm), is evaluated. To determine the protective ability of the protectant in the UVB wavelength range (<320 nm), this wavelength range can also be optionally evaluated.

In a further design of the invention, the evaluation of the protective ability of the protectant for light in a wavelength range from 400 nm to 500 nm is carried out in a method which is different from the evaluation of the protective ability of the protectant for light in a wavelength range from 280 nm to 400 nm. Due to its high absorption properties, human skin does not emit enough UVB radiation to measure the absorption spectrum of the applied product in the UVB range. It is therefore necessary to capture the absorption spectrum of the test material in the UVB part of the spectrum (280-320 nm) separately, using another technique. For this purpose, the in vivo remission spectrum in the wavelength range from 320 nm to 400 nm, the wavelength range from 280 nm to 320 nm, and the wavelength range from 400 nm to 500 nm are recorded with an in silico transmission spectrum.

In a further embodiment of the invention, the wavelength range of the irradiation wavelength or the wavelength range of the detection wavelength is smaller than the wavelength range of the evaluation wavelength. The wavelength ranges of the irradiation wavelength and the detection wavelength are preferably the same in a wavelength range of a maximum of 320 nm to 400 nm. The wavelength range of the evaluation wavelength comprises a maximum range of 280 nm to 500 nm.

In a further embodiment of the invention, the wavelength range of the irradiation wavelength or the wavelength range of the detection wavelength is smaller than the wavelength range of the evaluation wavelength. The wavelength ranges of the irradiation wavelength and the detection wavelength are preferably the same in a wavelength range of a maximum of 320 nm to 400 nm. The wavelength range of the evaluation wavelength comprises a maximum range of 280 nm to 500 nm.

In a further design of the invention, the wavelength range of the irradiation wavelength or the wavelength range of the detection wavelength in the wavelength range of the evaluation wavelength range is smaller than the wavelength range of the evaluation wavelength. The wavelength ranges of the irradiation wavelength and the detection wavelength are preferably equal in a wavelength range of 320 nm to 400 nm, wherein the wavelength ranges of the irradiation wavelength and the detection wavelength are smaller than the said wavelength range of 320 nm to 400 nm. In the wavelength range of the evaluation wavelength (maximum 280 nm to 500 nm), the wavelength range of the irradiation wavelength or the wavelength range of the detection wavelength in the wavelength range of the evaluation wavelength is smaller than the wavelength range of the evaluation wavelength.

In a further development of the invention, the wavelength range of the irradiation wavelength and/or the wavelength range of the detection wavelength is less than 100 nm, preferably less than 50 nm, and particularly preferably less than 25 nm. Therefore, only one beam source is needed to emit electromagnetic radiation, which has a small wavelength range of the irradiation wavelength. Similarly, to record the reflectance spectrum, a detector is needed that can detect a small wavelength range of the detection wavelength. The beam source and detector can therefore be designed to be cost-effective in production and operation.

In a further embodiment of the invention, the irradiation wavelength and/or the detection wavelength comprises only light with wavelengths outside the wavelength range of 400 nm to 450 nm. To capture an in vivo measurement, the UVA wavelength range (320 nm-400 nm) in particular is coupled into the measuring body.

In a further development of the invention, the irradiation wavelength and/or the detection wavelength comprises only light with wavelengths outside the wavelength range of 400 nm to 500 nm. In particular, the wavelength range of UVA and UVB is radiated. This wavelength range poses the greatest risk to human skin, so determining the protective ability of a protectant is particularly important.

In a further embodiment of the invention, the evaluation wavelength comprises wavelengths λ with λ<400 nm. To capture an in vivo measurement, in particular the UVA wavelength range (320 nm-400 nm) is evaluated.

In a further design of the invention, the evaluation wavelength comprises wavelengths λ with 320 nm<λ<400 nm. To capture an in vivo measurement to determine the protective ability of the protectant, in particular the UVA wavelength range (320 nm-400 nm) is evaluated. To determine the protective ability of the protectant in the UVB wavelength range (<320 nm), this wavelength range can also be optionally evaluated.

In a further design of the invention, the radiation is emitted in vivo onto the human skin. In the case of an in vivo measurement, the measuring body is the human skin covered with the protectant to be tested. The protection factor evaluation system is suitable for introducing electromagnetic radiation into a measuring body, preferably into the human skin, by means of the beam source arranged in the protection factor evaluation system. In addition, the protection factor evaluation system is suitable for detecting the remitted and/or transmitted electromagnetic radiation by means of the detector unit that is arranged in the protection factor evaluation system.

In a further advantageous design of the invention, the protective ability of the protectant is evaluated from two measurements. In a further development of the invention, a first measurement is carried out before the protectant is applied to the measuring body. In a further aspect of the invention, a second measurement is carried out after the protectant has been applied to the measuring body.

In a further embodiment of the invention, the beam source generates polychromatic radiation, wherein the generated polychromatic radiation is radiated unfiltered onto the measuring body. The beam source generates radiation in a maximum wavelength range of 280 nm to 500 nm (UVB to blue light). The generated polychromatic radiation is not altered by optical elements (filters, monochromators) in the generated wavelength range from the generation of the polychromatic radiation until it hits a measuring body. This results in a maximum intensity of the generated electromagnetic radiation, a likewise maximum intensity of the remitted or transmitted radiation, and consequently a high signal-to-noise ratio.

In a further design of the invention, a message is issued if the measurement results of the reference measurement deviate from the calibration measurement by more than a threshold value. The radiation is only emitted from the measuring head if the measurement results of the reference measurement deviate from the calibration measurement below a threshold value. This ensures the accuracy and reproducibility of the determination of the protection factor of a protectant.

Exemplary embodiments of the method according to the invention for determining a protection factor using a protection factor evaluation system are shown schematically in the drawings in a simplified manner and are explained in more detail in the following description.

In the drawings:

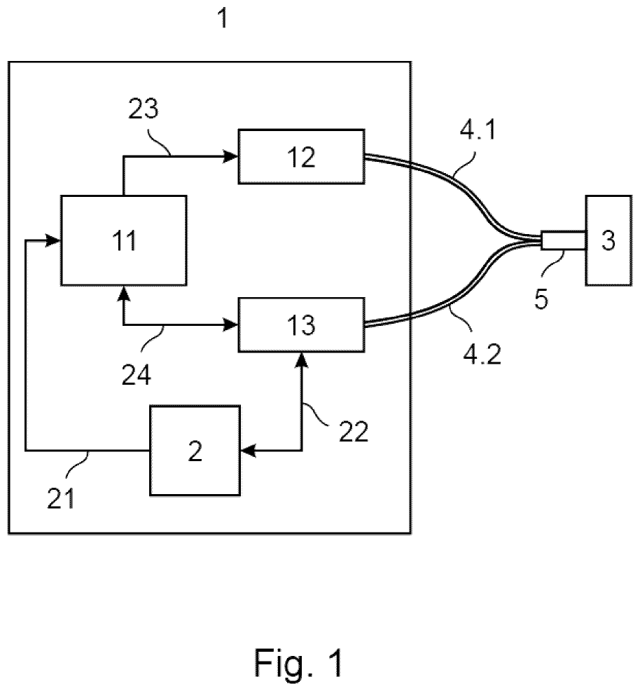

FIG. 1: shows a protection factor evaluation system

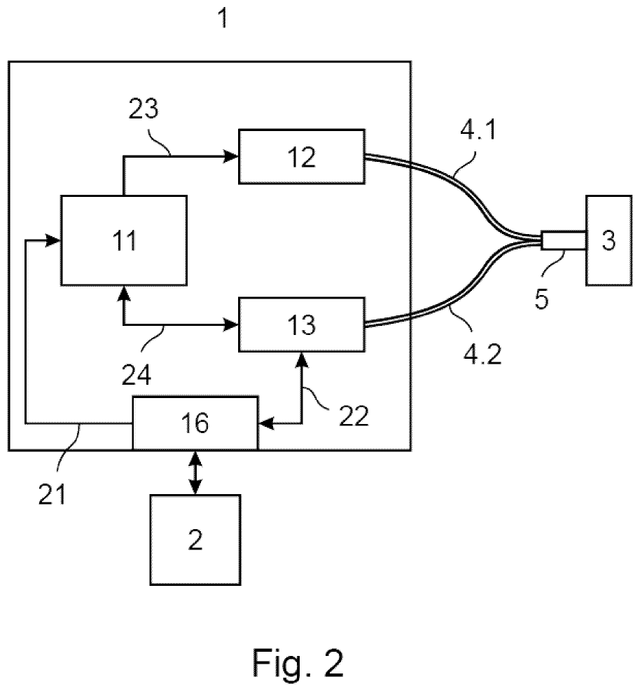

FIG. 2: shows the external control unit of a protection factor evaluation system

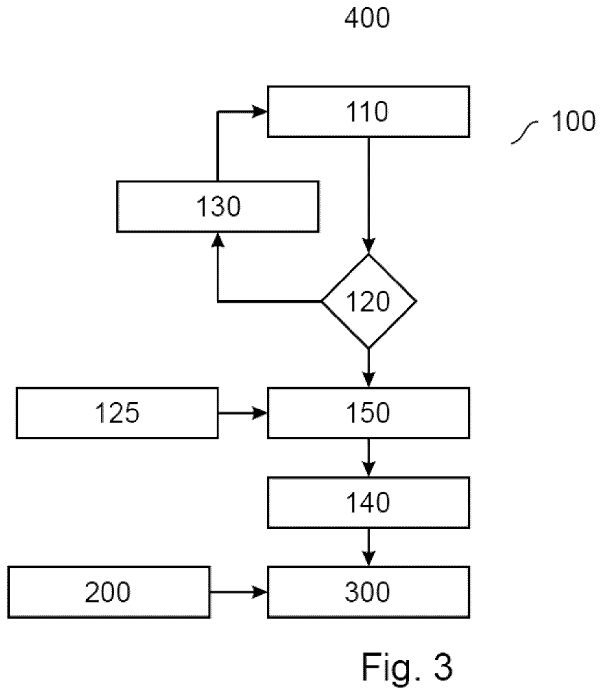

FIG. 3: shows a method for performing a reflection measurement to determine an in vivo reflection spectrum

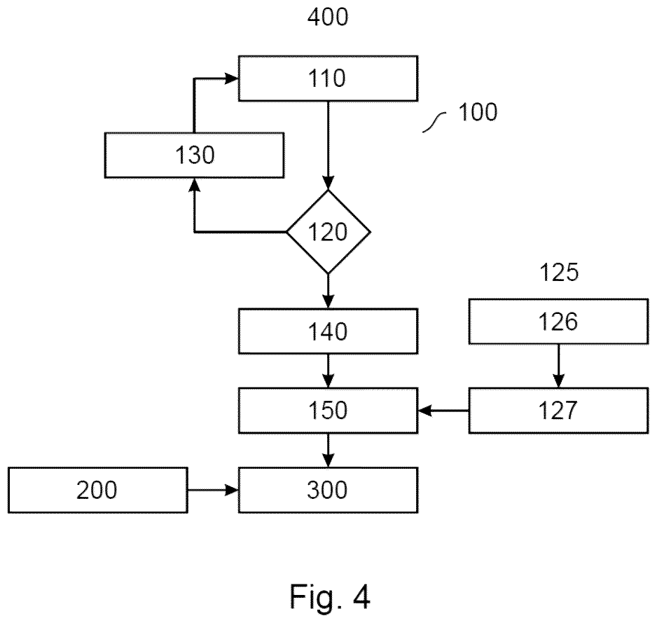

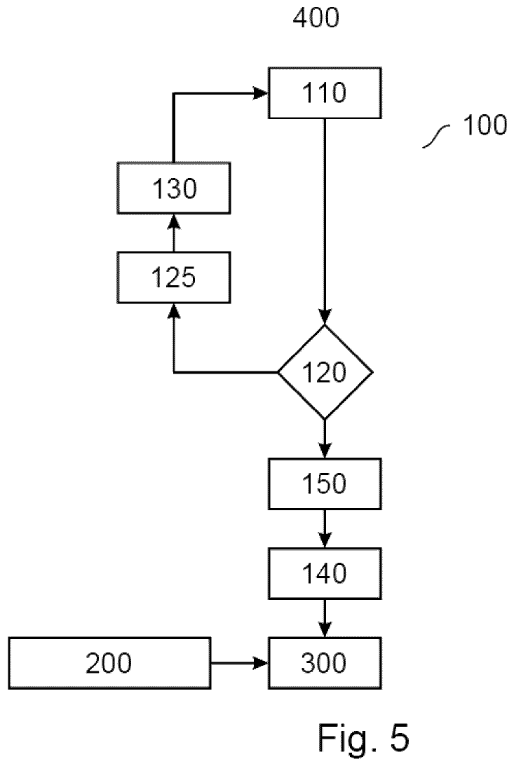

FIG. 4: shows a method for evaluating the protective ability of a protectant with a measurement to capture a reflection spectrum and a measurement to determine the ITA° value FIG. 5: shows a method for performing a reflection measurement to determine an in vivo reflection spectrum, ITA° correction value determination while carrying out the method

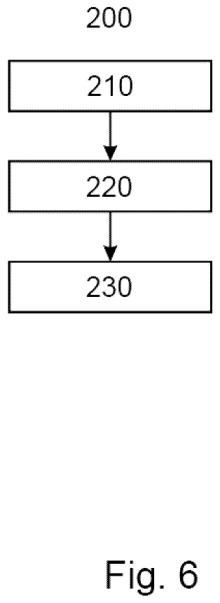

FIG. 6: shows a method for performing a measurement to determine a transmission spectrum

FIG. 1 shows schematically an exemplary embodiment of the protection factor evaluation system 1 according to the invention for carrying out an in vivo measurement. The protection factor evaluation system 1 has a beam source device 12. The radiation source device 12 has a beam source and optical elements which are intended and/or suitable for conditioning and/or redirecting the radiation generated by the beam source, such as light guides, filters, monochromators, mirrors, and/or other optical elements.

By means of a light guide 4.1, the spectrum emitted by the beam source device 12 is introduced into the measuring body 3 via the probe head 5, the light reflected by the measuring body 3 reaches the detector unit 13 via another light guide 4.2. The detector unit 13 has a monochromator, filter, photomultiplier, spectrometer, and/or a photodiode. In this and all subsequent embodiments, the detector unit 13 comprises a photodiode. The detector unit 13 and the beam source device 12 are connected via data lines 23, 24 to a beam source controller 11, which in turn is connected to the control unit 2 via another data line 21. The control unit 2 is usually a PC or notebook computer with a suitable computer program. The control unit 2 and the detector unit 13 are also connected to each other via a data line 22.

A further embodiment of the protection factor evaluation system 1 according to the invention is shown in FIG. 2. The protection factor evaluation system 1 also has the radiation source device 12. By means of the light guide 4.1, the light emitted by the beam source device 12 is introduced into the measuring body 3 via the probe head 5, the light reflected by the measuring body 3 reaches the detector unit 13 via another light guide 4.2. The detector unit 13 and the beam source device 12 are connected to a beam source controller 11 via data lines 23, 24. In this embodiment, the control unit 2 is arranged remotely from the protection factor evaluation system 1 and is connected thereto via the interface 16. The connection can be wired or wireless, e.g. via IP connection, Bluetooth, etc. The interface 16 and the detector unit 13 on the one hand and the interface 16 and the beam source controller 11 are connected to each other via the data lines 21, 22.

FIG. 3 shows an embodiment of an implementation of the method 100 of an in vivo measurement for capturing the remission spectrum by means of the protection factor evaluation system 1 according to the invention from the previous exemplary embodiments (see FIG. 1, FIG. 2). The method is carried out in vivo. The method 100 of a measurement requires the capture of a remission spectrum of the skin of test subject 3 untreated with protectant and of the skin treated with protectant.

For this purpose, the probe head 5 is applied to the untreated skin of test subject 3, that is, the protectant to be examined is not applied to the skin of test subject 3. For this purpose, a location on the inner side of the forearm or back of a test subject 3 is usually selected. The first measurement 110 is then carried out by the beam source controller 11 controlling the beam source device 12 such that the light generated by the beam source is guided through the light guide 4.1 onto the skin of test subject 3.

The light generated by the beam source is irradiated unfiltered onto the measuring body 3 to ensure a high S/N ratio. In particular, the light generated by the beam source is polychromatic with an intensity maximum at a wavelength in the UVA range of 340 nm. Irradiation occurs at an intensity that does not cause acute damage to the skin, which is below the simple MED, or below the MZB values, or significantly below the values caused by solar radiation. The light reflected from the skin of test subject 3 is guided through the light guide 4.2 to the photodiode of the detector unit 13, detected by the photodiode and converted into measured values, the measured values are sent to the control unit 2 and stored in the control unit 2.

The control unit 2 then asks from 120 whether the second measurement of the skin of test subject 3 treated with protectant has already been carried out. If this is not the case, the control unit 2 will indicate this. To carry out the second measurement 110 with applied protectant, the protectant is applied 130 to the skin of test subject 3 in the amount of 2.0 mg/cm² on the skin surface to be tested. The application 130 of the protectant and the subsequent second measurement 110 are carried out at the same location of the measurement sample 3, in particular at the same location on the skin of a test subject 3, to ensure the reproducibility of the first and second measurements 110. Also to ensure reproducibility, the control unit 2 controls the beam source controller 11 such that the beam source controller 11 controls the beam source of the beam source device 12 such that the light generated by the beam source is guided through the light guide 4.1 onto the skin of test subject 3, wherein the intensity and exposure time of the first and second measurements 110 match.

The light reflected from the skin of test subject 3 is likewise captured by the photodiode of the detector unit 13 and converted into measured values, the measured values are sent to the control unit 2 and stored in the control unit 2.

If the query 120 shows that the second measurement 110 has already been carried out, the ITA° value of the skin of test subject 3 is read in and the ITA° correction 150 is carried out. The ITA° value of the skin of subject 3 was determined in a separate measurement 125 prior to performing the method 100 of recording an in vivo reflectance spectrum. This is followed by an evaluation 140 of the reflection measurement. For this purpose, the control unit 2 executes a program to calculate the transmission spectrum T_{in vivo} according to equation 1:

$${T}_{i n vivo} = \frac{1}{S {F}_{i n vivo}} = \sqrt{\frac{R}{{R}_{0}}} \qquad Eq . 1$$

wherein T_{in vivo} is a function of the wavelength λ, SF_{in vivo} is the protection value determined by the in vivo method 100, R₀ is the reflected intensity of the untreated skin of test subject 3 as a function of the wavelength λ, R is the reflected intensity of the skin of test subject 3 treated with protectant as a function of the wavelength λ. The method 100 presented here requires a time from a few seconds to several tens of seconds.

To determine the protection factor, the protectant is evaluated in the next process step 300. For this purpose, the control unit 2 reads in a transmission spectrum that is calculated in silico or determined according to ISO 24443 (see FIG. 6).

If the amounts and properties of the UV filter substances of a protectant are known, the UV transmission can be calculated, taking into consideration the irregularity of the film and its photodegradation. Based on the simulated UV transmission, the protective ability of the protectant and all parameters that characterize the protection against UVA and/or UVB can be calculated in silico.

The in silico determination of the data of a transmission spectrum is carried out, for example, in a laboratory based on ISO 24443. An application of 2.0 g/cm² of protectant is assumed. In this embodiment, the emitted radiation is in the wavelength range from 280 nm to 500 nm (UVB to blue light). In further embodiments, the irradiation wavelength range can be in the wavelength range between 280 nm and 2000 nm and preferably between 280 nm and 800 nm.

The evaluated wavelength range of the transmission spectrum comprises the wavelength range from 280 nm to 2000 nm, preferably the wavelength range from 280 nm to 800 nm or particularly preferably the wavelength range from 280 nm to 500 nm and/or the wavelength range from 400 nm to 500 nm and/or the wavelength range from 400 nm to 450 nm. In this exemplary embodiment, the evaluation wavelength range of the transmission spectrum is 280 nm to 320 nm.

The in silico transmission spectrum data is stored in a database and can be accessed from the database at any time. To determine the protection factor of a skin protectant, the in silico transmission spectrum is loaded 200 into the evaluation unit 10 which is connected to the database via an Internet connection.

The results of the evaluations 140, 230 of the in vivo remission spectrum and the in silico transmission spectrum are evaluated 300 together using the evaluation unit 10. For this purpose, the hybrid transmission spectrum T_{hyb} is first calculated, wherein the in silico transmission spectrum T_{in silico} is scaled by means of the reflection spectrum T_{in vivo}:

$${T}_{hyb} ( λ ) = {T}_{i n silico} ( λ ) * \frac{{T}_{i n vivo} ( {λ}_{LED} )}{{T}_{i n silico} ( {λ}_{LED} )} \qquad Eq . 2$$

The protective ability of the protectant for the spectral range from UVA (320 nm) to the HEV spectral range (450 nm) SF then results according to equation 4 (here E=IPD(λ) is the IPD spectrum; S=I(λ) is the solar spectrum):

$$S F = \frac{{\int }_{320}^{450} S ( λ ) E ( λ ) d λ}{{\int }_{320}^{450} S ( λ ) E ( λ ) {T}_{hyb} d λ} \qquad Eq . 3$$

The protective ability of the protectant in the spectral range of blue light (400 nm to 500 nm) is determined according to equation 5 (here E=IPD(λ) is the IPD spectrum; S=I(λ) is the solar spectrum)

$$S F ( 400 nm - 500 nm ) = \frac{{\int }_{400}^{500} S ( λ ) E ( λ ) d λ}{{\int }_{400}^{500} S ( λ ) E ( λ ) {T}_{hyb} d λ} \qquad Eq . 4$$

The protective ability of the protectant from 400 nm to 450 nm SF (400 nm-450 nm) is determined according to equation 6 (here E=IPD(λ) is the IPD spectrum; S=I(λ) is the solar spectrum):

$$S F ( 400 nm - 450 nm ) = \frac{{\int }_{400}^{450} S ( λ ) E ( λ ) d λ}{{\int }_{400}^{450} S ( λ ) E ( λ ) {T}_{hyb} d λ} \qquad Eq . 5$$

The protective ability of the protectant for the spectral range of UVA (320-400 nm) UVA-SF then results according to equation 4 (here E=PPD(λ) is the PPD spectrum; S=I(λ) is the solar spectrum or UVA source for PPD-test):

$$UVA - SF = \frac{{\int }_{320}^{400} S ( λ ) E ( λ ) d λ}{{\int }_{320}^{400} S ( λ ) E ( λ ) {T}_{hyb} d λ} \qquad Eq . 6$$

The protective ability of the protectant for the spectral range from UVB to UVA (280-400 nm) UV-SF is then calculated according to equation 4

$$UV - SF = \frac{{\int }_{290}^{400} S ( λ ) E ( λ ) d λ}{{\int }_{290}^{400} S ( λ ) E ( λ ) {T}_{hyb} d λ} \qquad Eq . 7$$

In all equations for SF or UVA-SR, an optional correction function F (SF)=SF_corr or F (UVA-SF)=UVA-SF_corr is used, which describes differences depending on skin type, for example. A correction function makes values of different skin types comparable and outputs a corrected value SF_{corr} or UVA-SF_{corr}.

For example, F can be a linear factor (i.e. F(SF)=SF*C) or an exponential function (i.e. F(SF)=SF^{C}). C can depend on the skin type or the ITA° value, for example. As shown in Eq. 4, the formulas in Eq. 3 to 7 can be written as follows:

$$S {F}_{corr} = F ( S F ) = F ( \frac{{\int }_{320}^{450} S ( λ ) E ( λ ) d λ}{{\int }_{320}^{450} S ( λ ) E ( λ ) {T}_{hyb} d λ} ) \qquad Eq . 8$$

Since the in vivo value is measured without photodegradation, photodegradation should be appropriately taken into consideration. This can be done by calculating T__{in silico} with T_{in silico_irr}(λ) and without photodegradation and calculating a spectral quotient SRPD from it

$$SRPD ( λ ) = \frac{{T}_{i n silico \_ irr} ( λ )}{{T}_{i n silico} ( λ )} . \qquad Eq . 9$$

This will then be calculated

T_{hyb_irr}(λ)=T_{hyb}(λ)*SRPD(λ) Eq.10

The method can be calibrated using suitable reference methods such as electron spin resonance spectroscopy (ESR).

FIG. 4 shows an embodiment of the method 400 according to the invention for determining the protective ability of a protectant, wherein an ITA° correction 150 is carried out, which is carried out by means of a measurement 125 for determining the ITA° value. The method 100 of an in vivo measurement for capturing the remission spectrum is carried out in vivo. For this purpose, the probe head 5 is applied to the untreated skin of the test subject 3. Then the first measurement 110 is taken. The light reflected from the skin of test subject 3 is guided through the light guide 4.2 to the photodiode of the detector unit 13, detected by the photodiode and converted into measured values, the measured values are sent to the control unit 2 and stored in the control unit 2. The control unit 2 then asks from 120 whether the second measurement of the skin of test subject 3 treated with protectant has already been carried out. If this is not the case, the control unit 2 will indicate this. To carry out the second measurement 110 with applied protectant, the protectant is applied to the skin of test subject 3. The application 130 of the protectant and the subsequent second measurement 110 are carried out at the same location of the measurement sample 3, in particular at the same location on the skin of a test subject 3. The light reflected from the skin of test subject 3 is likewise captured by the photodiode of the detector unit 13 and converted into measured values, the measured values are sent to the control unit 2 and stored in the control unit 2.

If the query 120 shows that the second measurement 110 has already been carried out, an evaluation 140 of the reflection measurement takes place like in the previous exemplary embodiment (see FIG. 3). The ITA° value of the skin of test subject 3 is then read in and the ITA° correction 150 is carried out. The ITA° value of the skin of test subject 3 is determined in the separate measurement 125 and a colorimetric reflection measurement is carried out 126. For this purpose, a probe head is applied to the skin of subject 3 and white light is shined onto and into the skin of subject 3. The wavelength of the emitted white light is 440-670 nm. The light reflected from the skin of test subject 3 is captured and converted into measured values. In the evaluation 127 of the measurement 125, the measured values of the probe head are adjusted as closely as possible to the DIN standard values using a special color matrix and expressed as XYZ (tristimulus). The determined skin color is sent to the control unit 2 as an ITA° value in the CIELAB color space and is used to perform the ITA° correction 150.

Like in the above exemplary embodiment (see FIG. 3), the data of the transmission spectrum 200 (determined in vitro or in silico) are then read into the control unit 2, the evaluation and determination 300 of the protection factor of a protectant is carried out as described in FIG. 3.

An advantageous embodiment of the method 400 according to the invention is shown in FIG. 5. In this embodiment, the measurement 125 for determining the ITA° value of the skin of test subject 3 is carried out with the same protection factor evaluation system 1 with which the method 400 for evaluating the protective ability of a protectant is carried out. In addition, measurement 125 is performed to determine the ITA° value of the skin of test subject 3 during the method 400.

The method 100 of an in vivo measurement for capturing the remission spectrum is carried out in vivo as already described in FIG. 3 and FIG. 4. For this purpose, the probe head 5 is applied to the untreated skin of the test subject 3. Then the first measurement 110 is taken. The light reflected from the skin of test subject 3 is guided through the light guide 4.2 to the photodiode of the detector unit 13, detected by the photodiode and converted into measured values, the measured values are sent to the control unit 2 and stored in the control unit 2. The control unit 2 then asks from 120 whether the second measurement of the skin of test subject 3 treated with protectant has already been carried out. If this is not the case, the control unit 2 will indicate this.

Then measurement 125 is carried out to determine the ITA° value. For this purpose, the probe head 5 of the protection factor evaluation system 1 is placed on the same spot on the skin of a test subject 3. The control unit 2 controls the beam source controller 11 such that the beam source device 12 radiates white light with a wavelength range of 440 nm to 670 nm onto the skin of test subject 3. Alternatively, light in the UV range and/or visual range can be used. The light remitted by the skin of test subject 3 is captured, the control unit 2 determines an ITA° value of the skin of test subject 3. Then application 130 of the protectant and the second measurement 110 take place. The determined skin color is used as an ITA° value in the CIELAB color space to perform the ITA° correction 150. Like in the above exemplary embodiment (see FIG. 3), the data of the transmission spectrum 200 (determined in vitro or in silico) are then read into the control unit 2, the evaluation and determination of the protection factor of a protectant is carried out as described in FIG. 3.

An embodiment of an implementation of the method 200 of an in vitro transmission measurement is shown in FIG. 6. The in vitro measurement 200 is carried out using a measuring arrangement separate from the previous exemplary embodiment (see FIG. 1, FIG. 2) under laboratory conditions. A commercially available spectrometer with a xenon lamp is used, which emits and captures radiation in the wavelength range from 280 nm to 500 nm (UVB to blue light). The resolution of the device is 1 nm.

For this purpose, the protectant to be tested is applied 210 to a roughened PMMA plate in an amount of 1.3 mg per cm². The PMMA plate has a roughness of 5 μm. The transmission measurement and the subsequent evaluation 230, wherein the protection value for the entire wavelength range from 280 nm to 500 nm SF is determined according to equation 11, are then carried out 220:

$$S F = \frac{{\int }_{280}^{500} S ( λ ) E ( λ ) d λ}{{\int }_{280}^{500} S ( λ ) E ( λ ) {T}_{trans} d λ} \qquad Eq . 11$$

with S as solar spectrum, E as erythema effect spectrum, Tₜᵣₐₙₛ as transmission and dλ as wavelength increment (1 nm).

### LIST OF REFERENCE NUMERALS

1 protection factor evaluation system
2 control unit
3 sample/measuring body
4.1, 4.2 light guide/fiber bundle
5 probe head
11 beam source controller
12 beam source device
13 detector unit/spectrometer
21 connection beam source controller-control unit
22 connection detector unit/spectrometer-control unit
23 connection beam source controller-beam source device
24 connection beam source controller-detector unit
100 method for recording a reflection spectrum
110 carrying out a measurement of a reflection spectrum
120 query
125 measurement to determine the ITA° value
126 colorimetric reflection measurement
127 evaluation of the colorimetric reflection measurement
130 application of the protectant
140 evaluation of the reflection measurement
150 ITA° correction
200 method for recording a transmission spectrum
210 application of the protectant
220 carrying out a measurement of a transmission spectrum
230 evaluation of the transmission measurement
300 evaluation of the protectant
400 method for examining the protective ability of protectants

1. A method (400) for determining a protection factor of a skin protectant, comprising the steps of:
emitting (110) radiation from a radiation source unit (12) of a protection factor evaluation system (1),
detecting remitted radiation from a detector unit (13) of a protection factor evaluation system (1),
transferring data of the remitted radiation to an evaluation unit (10),
evaluating (300) the protection factor in an evaluation wavelength range,
wherein a correction function is taken into consideration when evaluating (300) the protection factor, wherein the correction function describes differences depending on the skin type.

2. The method (400) for determining a protection factor of a skin protectant according to claim 1,
characterized in that the correction function outputs a corrected value SF_{corr} or UVA-SF_{corr}.

3. The method (400) for determining a protection factor of a skin protectant according to claim 1,
characterized in that
the correction function is F (SF)=SF__{corr} or F (UVA-SF)=UVA-SF__{corr}.

4. The method (400) for determining a protection factor of a skin protectant according to claim 1,
characterized in that
the correction function makes the measured values of different skin types comparable by the corrected values.

5. The method (400) for determining a protection factor of a skin protectant according to claim 1,
characterized in that
the correction function F is a linear factor or an exponential function.

6. The method (400) for determining a protection factor of a skin protectant according to claim 1,
characterized in that
the correction function has a constant C,
wherein C depends on the skin type or the ITA° value.

7. The method (400) for determining a protection factor of a skin protectant according to claim 1,
characterized in that
the skin type-dependent differences are determined by a measurement (125).

8. The method (400) for determining a protection factor of a skin protectant according to claim 7,
characterized in that
the skin type-dependent differences are determined by a reflection measurement.

9. The method (400) for determining a protection factor of a skin protectant according to claim 1,
characterized in that
the emitted radiation comprises an irradiation wavelength range between 280 nm and 2000 nm, preferably 280 nm to 800 nm, particularly preferably the range from 280 nm to 500 nm.

10. The method (400) for determining a protection factor of a skin protectant according to claim 9,
characterized in that
the evaluation wavelength range is different from the irradiation wavelength range.

11. The method (400) for determining a protection factor of a skin protectant according to claim 1,
characterized in that
the protection factor of the protectant is evaluated (300) from the remitted radiation and a transmission spectrum.

12. The method (400) for determining a protection factor of a skin protectant according to claim 11,
characterized in that
the data of the transmission spectrum for determining the protection factor is read from a database of the evaluation unit (10).

13. The method (400) for determining a protection factor of a skin protectant according to claim 11,
characterized in that
the transmission spectrum data for determining the protection factor is in silico data.

14. The method (400) for determining a protection factor of a skin protectant according to claim 11,
characterized in that
the wavelength range of the transmission spectrum includes the evaluation wavelength range.

15. The method (400) for determining a protection factor of a skin protectant according to claim 1,
characterized in that
the evaluation wavelength range of the transmission spectrum comprises the wavelength range from 280 nm to 2000 nm, preferably the wavelength range from 280 nm to 800 nm, or particularly preferably the wavelength range from 280 nm to 500 nm and/or the wavelength range from 400 nm to 500 nm and/or the wavelength range from 400 nm to 450 nm.

16. The method (400) for determining a protection factor of a skin protectant according to claim 1,
characterized in that
the evaluation (300) of the protective ability of the protectant for light in a wavelength range from 400 nm to 500 nm is carried out in a method separate from the evaluation (300) of the protective ability of the protectant for light in a wavelength range from 280 nm to 400 nm.

17. The method (400) for determining a protection factor of a skin protectant according to claim 1,
characterized in that
the evaluation (300) of the protective ability of the protectant for light in a wavelength range from 400 nm to 500 nm is carried out in a method different from the evaluation (300) of the protective ability of the protectant for light in a wavelength range from 280 nm to 400 nm.

18. The method (400) for determining a protection factor of a skin protectant according to claim 1,
characterized in that
the wavelength range of the irradiation wavelength or the wavelength range of the detection wavelength is smaller than the wavelength range of the evaluation wavelength.

19. The method (400) for determining a protection factor of a skin protectant according to claim 1,
characterized in that
the wavelength range of the irradiation wavelength or the wavelength range of the detection wavelength in the wavelength range of the evaluation wavelength range is smaller than the wavelength range of the evaluation wavelength.

20. The method (400) for determining a protection factor of a skin protectant according to claim 1,
characterized in that
the range of the irradiation wavelength and/or the wavelength range of the detection wavelength is less than 100 nm, preferably less than 50 nm, and particularly preferably less than 25 nm.

21. The method (400) for determining a protection factor of a skin protectant according to claim 1,
characterized in that
the irradiation wavelength and/or the detection wavelength only comprises light with wavelengths outside the wavelength range of 400 nm to 450 nm.

22. The method (400) for determining a protection factor of a skin protectant according to claim 1,
characterized in that
the irradiation wavelength and/or the detection wavelength only comprises light with wavelengths outside the wavelength range of 400 nm to 500 nm.

23. The method (400) for determining a protection factor of a skin protectant according to claim 1,
characterized in that
the evaluation wavelength comprises wavelengths outside the wavelength range of 400 nm to 500 nm.

24. The method (400) for determining a protection factor of a skin protectant according to claim 1,
characterized in that
the evaluation wavelength comprises wavelengths λ with λ<400 nm.

25. The method (400) for determining a protection factor of a skin protectant according to claim 1,
characterized in that
the evaluation wavelength comprises wavelengths λ with 320 nm<λ<400 nm.
